(19) United States
(12) Patent Application Publication
(10) Pub. No.: US 2019/0313934 A1
(43) Pub. Date: Oct. 17, 2019

(54) DEVICE AND SYSTEM FOR MONITORING AND TREATING MUSCLE TENSION-RELATED MEDICAL CONDITIONS

(71) Applicant: Biotrak Health, Inc., Wilmington, DE (US)

(72) Inventors: Eric Lee, Wilmington, DE (US); Robert L. Bratzler, Wilmington, DE (US); Erk Lillydahl, Wilmington, DE (US); Adam Kirell, Wilmington, DE (US)

(21) Appl. No.: 16/308,777

(22) PCT Filed: Jun. 12, 2017
(86) PCT No.: PCT/US2017/037054

§ 371 Date: Dec. 10, 2018

Related U.S. Application Data
(60) Provisional application No. 62/348,735, filed on Jun. 10, 2016.

(51) Int. Cl. A61B 5/0488; A61B 5/00; A61B 5/0205; A61B 5/01; A61B 5/0402; A61B 5/0476; A61B 5/1455; A61K 31/216; A61K 31/612; A61K 31/192

(52) U.S. Cl. CPC A61B 5/0488; A61B 5/02405; A61B 5/746; A61B 5/486; A61B 5/4848; A61B 5/7275; A61B 5/0022; A61B 5/7465; A61B 5/4839; A61B 5/02055; A61B 5/01; A61B 5/0402; A61B 5/0476; A61B 5/14551; A61K 31/216; A61K 31/612; A61K 31/192; A61B 5/7405; A61B 5/742; A61B 5/7455; A61B 5/6803

(57) ABSTRACT

Mobile systems and devices can employ surface electromyography (sEMG) technology and other sensing technologies for measuring muscle tension and managing chronic pain conditions. These systems and devices can sense and quantify excessive muscle tension and can facilitate management of excessive muscle tension and chronic pain through cognitive behavioral therapy, pharmacologic intervention, or a combination of both.

### BACKGROUND OF THE INVENTION

Excessive muscle tension has been identified as a leading cause of chronic tension-type headaches, migraine, orofacial pain, as well as other pain conditions remote to the head and neck, such as low back pain, chronic pelvic floor pain, fibromyalgia, etc. The most common treatment option involves the use of pharmacologic agents (or drugs, which term is used synonymously in the present invention). Studies have also shown that behavioral modification therapies including cognitive behavioral therapy (CBT), biofeedback, and relaxation training are effective in mitigating or resolving these muscle tension-related syndromes and a myriad of other medical conditions associated with mental stress and anxiety, including depression, anxiety, multiple sclerosis, hypertension, insomnia, irritable bowel syndrome, attention deficit hyperactivity disorder (ADHD), and urinary incontinence, etc. Traditionally, behavioral modification treatment of these conditions is done in a psychologist's office using instruments and wired biosensors connected to the patient. Close supervision and biofeedback-based coaching by trained professionals generally yield significant improvement and alleviation of these muscle-tension-induced conditions. This approach, however, has not attained its potentially widespread use because of inconvenience, expense, and stigma associated with psychological treatments. The vast majority of patients referred to a psychology office for biofeedback simply never show up there. Sporadic or unreliable access to professional resources also contributes to inconsistent outcomes. The economic and social costs of the resulting failure of compliance are considerable in terms of increased direct medical costs, lost productivity, and lower quality of life.

As an example, migraine and tension-type headaches (TTHA) are among the most pervasive, often debilitating, tension-related pain conditions (see “Headache Disorders,” Fact Sheet updated April 2016, World Health Organization (website: http://www.who.int/mediacentre/factsheets/fs277/en/). In the United States alone, more than 38 million people suffer from these conditions as of 2016 (see Russo A F. Calcitonin gene-related peptide (CGRP): a new target for migraine. Annual Review of Pharmacology and Toxicology. 2015; 55:533-552; see also Migraine facts. Migraine Research Foundation website. http://migraineresearch foundation.org/about-migraine/migraine-facts/). A recent study in Europe also associated headache with stress (see Schramm, S H, et al., The association between stress and headache: A longitudinal population-based study, Cephalalgia, 2015, Vol. 35(10) 853-863). These conditions are most often treated using drugs as a primary therapeutic approach. However, it is also well established that biofeedback therapies can be used to alleviate the underlying stress and tension that cause or exacerbate such disorders (see Yucha, C. and Montgomery D., “Evidence-Based Practice in Biofeedback and Neurofeedback,” Association for Applied Psychophysiology and Biofeedback, 2008, ISBN 1-887114-19-X). The present invention builds upon the paradigm that any physiological process that responds to stress will respond to stress reduction. Accordingly, one aspect of the invention relates to the effective reduction of tension-related pain conditions by means of biofeedback-assisted relaxation therapy (BART). Another aspect of the invention relates to the additive and/or synergistic effects of BART and administration of drugs, in the form of “combination therapy,” to produce treatment outcomes superior to those of either therapy used alone as “monotherapy.”

Advances in electronics and sensor technology have produced a variety of stationary (fixed in one location) and wearable (mobile) systems that monitor different body functions such as heart rate, repetitive motion (e.g., steps), blood pressure, blood oxygen level, respiration rate, etc. Certain systems combine one or more sensors and electronics for simultaneous monitoring of EEG (electroencephalogram), EKG/ECG (electrocardiogram), GSR (galvanic skin response), temperature, heart rate, heart rate variability (HRV) and other biological signals. As an example, a commercially available wearable device such as Muse™ is a meditation aid that offers real-time EEG-based biofeedback, but does not offer coaching nor is it directed towards treatment of a medical condition. These omissions limit its usefulness and effectiveness. Other digital therapeutic approaches target pre-diabetes (e.g., WellDoc, Glooko, Omada Health, Livongo), cardiovascular disorders and hypertension (AliveCor, Twine Health), and mental health (Akili, Ginger.io). These are either based on mobile software applications or a combination of mobile applications and live coaching, and do not rely on biosensor-based feedback. In the area of migraine headaches, commercial devices have recently become available based on electrostimulation. For example, Cefaly™ (U.S. Pat. No. 8,702,584) operates by external trigeminal nerve stimulation (e-TNS) where electrical pulses are sent to the upper branch of the trigeminal nerve to suppressing migraine attacks. eNeura™ (U.S. Pat. No. 8,740,765) uses single-pulse transcranial magnetic stimulation (sTMS) to manage migraine episodes. Thync™ (U.S. Pat. No. 8,903,494) is another system that uses electrical stimulation to activate nerve pathways for controlling stress level, mood, and sleep quality. gammaCore™ (U.S. Pat. No. 8,676,33) is a device based on non-invasive vagus nerve stimulation (nVNS) that uses electric current for transcranial stimulation. The term non-invasive refers in this case to the use of skin-contact electrodes in place of implantable electrodes. By definition, however, all electrostimulation methods are invasive. Long-term side effects will require extended monitoring and analysis to detect and establish conclusively. This contrasts with approaches based on non-invasive measurements of biological signals and treatment methods derived from analysis of those signals. Surface electromyography, or sEMG, detects electrical signals on the skin surface originating from muscle contractions. This is a non-invasive technology that has been relied upon by healthcare professionals in clinical settings for measuring muscle tension, and to manage tension-related stress and pain conditions. And yet, to this point, no mobile sEMG-sensor-based systems have been used diagnostically to identify the sufferers of muscle tension related syndromes who are candidates for BART used alone (monotherapy), or for synergistic benefits created by combining the use of relaxation training techniques and pharmacological treatment (combination therapy). There is also no known mobile sEMG-sensor-based system that alerts users of impending onset of muscle-tension-induced pain, and the opportunity to administer pharmacologic agents to avert full-blown pain. There is also no known mobile system to monitor muscle tension to measure the effect of a drug designed to treat excessive muscle-tension-induced medical conditions. There is also no known mobile system which combines sEMG biosensors with other biosensors in the same mobile system. Moreover, there is no known mobile system which measures, records, and stores sEMG signals with other biological signals for integrated data analyses on the mobile system and in computing devices remote to the mobile system. To reach these functional goals, there is a need for systems that directly measure patient status and provide ambulatory, real-time feedback and alerts to the user, in addition to self-guided coaching (or interactive coaching prescribed by a healthcare provider) to provide timely relief of the pain conditions, in addition to connectivity to enterprise-level data management and data analytics aimed at improving quality of care and patient outcome on an on-going basis.

The present invention, Halo™, is the first system that enables accurate sEMG measurements to be used as the basis for treatment protocols, especially BART, on a mobile technology platform familiar to modern users. Deployed as monotherapy or as drug combination therapy, it is aimed at greatly expanding the reach of healthcare professionals to a large patient population beyond the clinic, while reducing the cost of pain management by empowering users to self-administer proven behavioral and/or pharmacologic intervention procedures, or under the guidance of healthcare professionals, in a timely manner critical to successful outcomes.

### SUMMARY OF THE INVENTION

The present invention relates generally to systems and devices that employ surface electromyography (sEMG) technology for measuring muscle tension. These systems and devices can sense and quantify excessive muscle tension and can facilitate the reduction of tension through cognitive behavioral feedback therapy, e.g., relaxation training, pharmacologic intervention, or a combination of both. The present invention can therefore serve as an enhancement of, or alternative to, drug-based therapies and is therefore particularly relevant to the emerging field of digital therapeutics and integrated healthcare.

The present invention addresses many of the problems that exist in the prior art by offering a mobile version of the laboratory (stationary) sEMG instrument that monitors muscle tension in real time, is simple to use, and provides a meaningful indication of the user's condition as well as a selection of coached, well-established relaxation techniques. The portable system enables timely access and intervention in various situations. With convenient connectivity options, the present invention enables telemedicine and other modern treatment modalities previously considered too complicated or expensive to implement to a large patient population. As such, the present invention can contribute significantly to the new connected health paradigm.

In some embodiments, the present invention can comprise a wearable device that includes sensors for measuring one or more biological signals including muscle tension, using sEMG; brain activity, using EEG; heart activity, using EKG/ECG; blood flow, using pulse oximetry; blood vessel dilation using temperature sensors, and electrodermal activity using GSR. The device can also include electronics for transmitting output of the sensors to a computing device, where the output can be analyzed and displayed to the user, or analyzed, stored, and accessed to inform and support decisions on intervention and/or treatment in settings outside the clinic. In the current context, “computing device” refers generally to devices or systems with data processing capabilities and interfaces for interaction with users, which include patients and healthcare professionals. Some examples of computing devices are smartphones, desktop computers, laptop computers, tablets, and the like; and application-specific programs (“apps”) designed to operate on those devices. The apps can be configured to analyze the sensor output to identify when the user is experiencing excess muscle tension and other conditions, and in response, can notify the user to provide guidance on how to reduce the excess tension. In this way, the present invention can assist the user in reducing their stress-induced muscle tension and preventing the onset of an acute and/or chronic pain condition.

The app can also be configured to continue analyzing sensor output while and after presenting guidance to the user to thereby track the effectiveness of the presented tension reduction techniques, as well as treatment reminders, event logs, and other means of encouraging compliance to recommended activities. For example, the app may monitor sensor output while coaching the user through a relaxation technique and/or after recommending that a particular pharmacologic agent be administered to the user to verify whether the user's muscle tension subsides in response to these treatments. In this way, the app may learn over time which techniques and pharmacologic agents are most effective for the particular user and can customize itself for future recommendations. Similarly, the app may analyze historical sensor output to detect patterns or trends in the occurrence of muscle tension and subsequent pain conditions. For example, the app may detect that the user frequently experiences muscle tension at a particular time of day or at a particular location (e. g., by correlating GPS and other environmental data with the sensor output). The app may then incorporate such findings into future analysis to thereby allow the app to more accurately predict the onset or occurrence of muscle tension and pain. In other embodiments, the app may incorporate input from other sensors in the smartphone or other computing device such as accelerometers, altimeters, ambient light sensors, GPS coordinates, heart rate and the like, to further assist in the identification of environmental factors that may trigger or exacerbate muscle tension related conditions in addition to other triggers from emotional or physical stress, certain foods, hormones, and medical conditions (see http://www.migraine.org.uk/js/plugins/filemanager/files/downloads/Migraine_triggers.pdf).

In some embodiments, because mental stress is often manifested by tension in the muscles in the head, shoulders, and neck, the wearable device may be in the form of a patch, headband, hat, cap, helmet, hard hat, visor, neckband, headphone, headset, in-ear monitor, ear muffs, or shoulder strap that includes sensors for detecting the tension of muscles in the forehead, the temple region, the mandible region, the neck region, or other muscle groups in the head. For example, in one instance a neckband includes one or more external sensors that contact the trapezius muscles in the neck and/or shoulder region of the user. In other embodiments, the wearable device is configured to be worn on other parts of the body to allow the tension of any muscle or muscle group to be monitored. In this way, the present invention can be employed not only to monitor tension due to mental stress, but to also monitor desirable muscle tension such as in the case of athletic performance monitoring. In short, the present invention can be employed to monitor the tension of any muscle or muscle group to allow real time analysis and feedback.

In some embodiments, the present invention may employ additional types of sensors in addition to the sEMG sensors to detect other physiological parameters. In such cases, the wearable device or devices can be configured to relay output from any of the sensors to the app for analysis. In this way, the present invention can further analyze whether another physiological condition (e.g., blood oxygen level, heart rate) may be affecting muscle tension in either a positive or negative way.

In some embodiments, the present invention may employ a single sensor that is capable of detecting multiple biological electrical signals that traverse a broad frequency range and encompass traditional EEG, EKG/ECG and sEMG outputs. In such cases, the wearable system or devices can be configured to process the electrical signals to extract relevant EEG, EKG/ECG, and sEMG components for separate display and analysis.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

Understanding that these drawings depict only typical embodiments of the invention and are not therefore to be considered limiting of its scope, the invention will be described and explained with additional specificity and detail through the use of the accompanying drawings in which:

FIG. 1 illustrates an example of a system that includes a wearable device in the form of a headband and an app;

FIG. 2 illustrates the arrangement of electronic circuits, power supply, and sensors in the headband portion of the system.

FIG. 3 illustrates a simplified schematic of a computing device and mobile application.

FIG. 4 illustrates the network architecture of a mobile system comprising a wearable headband, computing device, and cloud-based components.

### DETAILED DESCRIPTION OF THE INVENTION

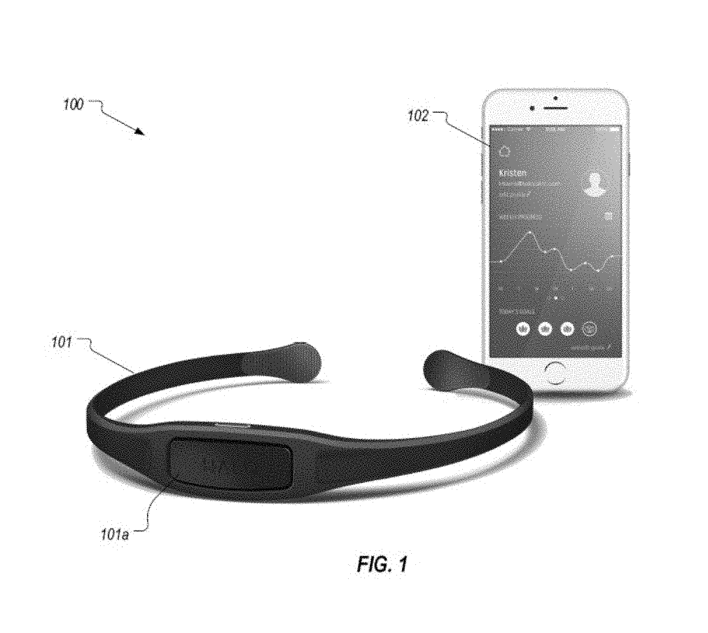

FIG. 1 illustrates an example system 100 in accordance with embodiments of the present invention. System 100 includes a wearable device 101 (which in this example is in the form of a headband) and an app 102 that is configured to communicate with wearable device 101. Device 101 comprises a computing unit 101a that includes a sensor unit which in this case is configured to detect a level of tension in the muscles of the forehead. Computing unit 101a can also include appropriate circuitry for transmitting an output of the sensor unit to app 102. In some embodiments, computing unit 101a may also include, or be coupled to, a feedback unit incorporated into device 101. The feedback unit can be configured to provide a user alert comprising at least one of audible, visual, and/or haptic feedback such as when the output of the sensor unit indicates an excessive level of tension. As used herein, the term “haptic” is understood to include any output that is detectable to a user via the touch sense. Non-limiting examples of haptic outputs may include vibration, heat, or electrical current.

The sensor unit of device 101 can employ surface electromyography sEMG to detect the tension level of the surrounding muscle(s). Electromyography is a technique for sensing a voltage that is generated when muscles contract. The voltage level produced during contraction is dependent upon the amount of muscle contraction as well as the number of contracted muscles. Therefore, if the muscles in the forehead are tightly contracted, a higher voltage will be produced than when the same muscles are only lightly contracted. By placing electrodes in close vicinity to the contracted muscle(s) (e.g., directly on top of these muscles), the voltage generated during contraction of the muscles can be detected. If the detected voltage rises above a specified threshold, it can be determined that the user is experiencing excess tension which may indicate the onset of a pain condition.

Computing unit 101a can be configured to continuously or periodically transmit the output of the sensor unit to app 102. App 102 can then store this output as an indication of the tension level of the monitored muscle(s) over time. App 102 can also be configured to provide guidance or coaching to the user when the output of the sensor unit indicates that the user is experiencing excess tension. For example, when the output of the sensor unit indicates that the user is experiencing excess tension in the muscles of the forehead, app 102 can be configured to guide the user through one or more relaxation exercises that are intended to reduce tension in the forehead and/or to take one or more pharmacologic agents. In this way, system 100 can assist the user in preventing or minimizing the occurrence of a tension headache.

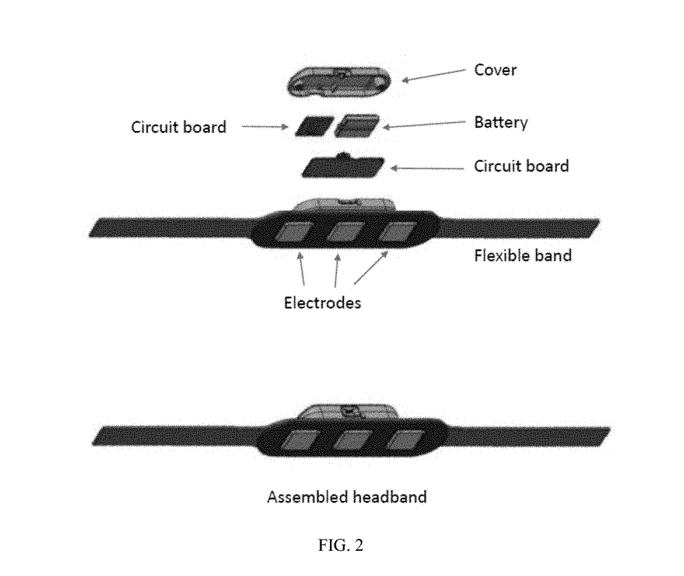

Computing device 101a may include an array of sensors for measuring muscle tension via sEMG, electronics (e. g., an analog to digital converter(s), amplifier(s), a processor), wireless communication capabilities (e.g., Bluetooth Low Energy (BLE)), an antenna, user-interface components for providing haptic, audio, and/or visual output from the wearable device, switches, and a power source in the form of a rechargeable battery. The main function of the wearable device is to capture sEMG signals generated by the subject and to transmit them to the computing device in a suitable format. A representative arrangement of components for the wearable device in the form of a headband is shown in FIG. 2.

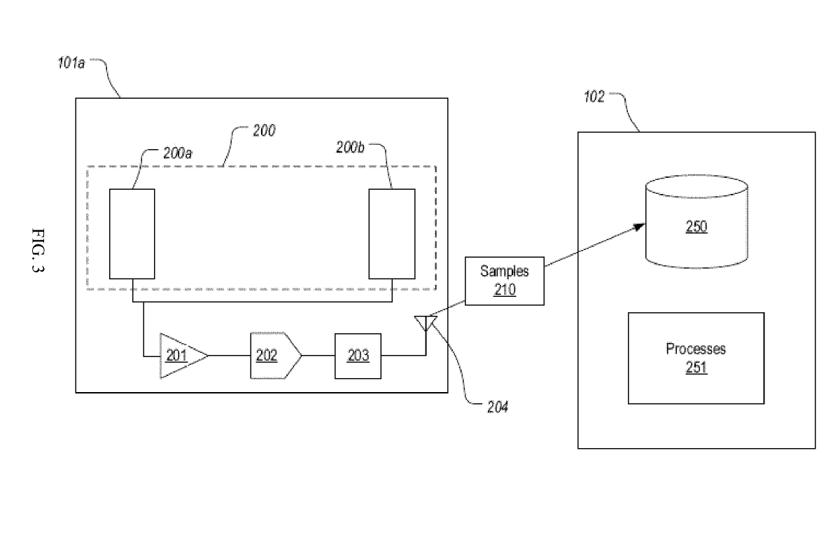

FIG. 3 provides a simplified schematic of computing device 101a to represent how sensor output can be provided to app 102. As shown, computing unit 101a can include a sensor unit 200 that comprises an array of electrodes/sensors (e.g., electrodes 200a and 200b) which are configured to sense a voltage level (e. g., the voltage that is generated by contracted muscles that are in the vicinity of electrodes 200a and 200b in accordance with sEMG techniques). Sensing unit 200 can be configured to continuously output a signal representing the sensed voltage level. In exemplary embodiments, this signal can be passed through an amplifier 201 and an analog to digital converter 202 and possibly filtered prior to being input to a transceiver 203. In some embodiments, transceiver 203 can represent a BLE module or another module that employs any suitable wireless protocol for communicating with a personal computing device such as a smartphone. Transceiver 203 can process the input signal in a suitable manner (e.g., to generate a number of samples 210 per time period) and then periodically transmit samples 210 to app 102.

Upon receiving samples 210 (e. g., via a Bluetooth interface provided by a computing device on which app 102 executes), app 102 can store the samples 210 in database 250. Alternatively, or additionally, app 102 may be configured to relay samples 210 to one or more remote computing devices (e. g., a server) for storage and/or analysis. Therefore, database 250 can generally represent a database on the computing device or on a server accessible to the app.

App 102 can include one or more processes 251 that are configured to further process samples 210 to output meaningful data to the user. For example, processes 251 may generate a display that indicates a tension level based on the values of samples 210. This tension level can be represented by normalizing the sensor output in accordance with a scale (e.g., a pain scale of 1-10), or depicted qualitatively to indicate the direction and magnitude of change. In some embodiments, processes 251 may be configured to monitor samples 210 over a period of time to calibrate system 100 for a particular user. For example, processes 251 can track minimum, maximum, and average values of samples 210 to identify a relaxed and a tension threshold for the user. In one embodiment, a tension threshold is set by an average value of samples 210 over a specific duration, for example 10 seconds. In some embodiments, processes 251 can be configured to prompt the user for input to identify when the user is in a relaxed or tense state thereby allowing processes 251 to correlate particular values of samples 210 with such states. In this way, app 102 is programmable to recognize when the user is in a tense state. In one embodiment, app 102 is programmable to include a custom user threshold, which is determined and set by the user.

In some embodiments, computing unit 101a may be configured to generate signals that include an identifier of a location of the body where the sensor unit is to worn. For example, assuming sensing unit 200 is intended to be worn on the forehead, transceiver 203 may be configured to associate a forehead identifier with samples 210 to thereby inform app that samples 210 represent the tension of the muscles in the forehead. Alternatively, if sensing unit 200 were intended to be worn over the temples, transceiver 203 may be configured to associate a temple identifier with samples 210. In embodiments where a device may be configured to be worn on various parts of the body, app 102 may be configured to allow the user to specify where the device is being worn. For example, processes 251 may be configured to receive input from the user identifying where the device is being worn and may then associate an appropriate location identifier with subsequently received samples. In one embodiment, processes 251 further comprise impedance detection to ensure proper contact and/or placement of one or more sensors and/or electrodes of the device. In other embodiments, sensors incorporate identifiers associated with specific locations of use and which are recognized automatically by processes 251 with no user intervention. One reason for associating samples with a location of the body that is being monitored for tension is to allow processes 251 to present appropriate guidance to the user when tension is detected. For example, if a device is being worn on the arm to measure tension in the bicep, app 102 may output different guidance/analysis when tension is detected than would be provided when the device is worn on the forehead. Accordingly, the association of a location indicator with samples can facilitate presenting appropriate guidance for assisting a user to reduce tension, properly train the muscles, or address some other condition.

In addition to the real-time monitoring and detection techniques described above, app 102 can also be configured to process historical samples to detect tension patterns, to identify which guidance techniques produced better results, or to detect a potential condition. For example, processes 251 can analyze samples stored in database 250 (which can be stored with a timestamp) to determine whether the user experiences tension at a particular time of day. If so, app 102 can notify the user and assist the user in determining why tension is occurring at that particular time. For example, if device 101 is worn at night and includes a sensor unit positioned over the temporomandibular joint, app 102 may detect that the user experiences tension in the temporomandibular joint region, such as in the masseter muscle, while sleeping and may present recommendations to the user to address the issue.

In some embodiments, app 102 may correlate data from external sensors, for example, location data, with sensor data 210. For example, while receiving sensor data 210, app 102 may also obtain current GPS data. Then, app 102 can process sensor data 210 to identify times of relaxation or tension and correlate these times with the user's location. In this way, app 102 can assist the user in identifying situations/locations when the user is likely to experience tension and/or relaxation.

App 102 may also monitor samples over time to identify whether a user is improving or regressing. For example, app 102 may evaluate samples generated at a particular time or at a particular location over a number of days or visits to that location. If the samples indicate a reduction in tension over time, app 102 can notify the user that current treatment techniques appear to be working. In contrast, if the samples indicate an increase or no change in tension, app 102 may recommend other treatments/techniques. In short, app 102 can be configured to evaluate obtained samples to not only identify when the user is currently experiencing tension, but to also identify patterns in occurrences of tension, correlations of occurrences with locations or situations, trends in occurrences, etc. In this way, app 102 can assist the user in identifying and implementing an appropriate treatment technique.

In some embodiments, App 102 may include features to capture, record, and document the condition of the user, including but not limited to headache conditions in terms of frequency, intensity, and duration, and to store such information before, during, or after treatment. In one embodiment, the condition information is entered in the form of a “headache diary.” In some embodiments, App 102 may include features to query, record, and document the administration of one or more pharmacologic agents that the user self-administers or is administered by a healthcare provider as treatment for the muscle tension or headache conditions, including the identity of the pharmacologic agent or agents, dosage, and frequency of administration, undesirable side effects, and the therapeutic effect the user experiences in terms of frequency, intensity, and duration of the muscle tension or headache conditions. In some embodiments, App 102 may include features to capture, record, and document the condition of the user before, during, and after the concurrent, contemporaneous but intermittent, periodic, or sequential use of the present invention with one or more pharmacologic agents, also referred to as combination therapy. Such information recorded over time is part of the wellness record, health record, or medical record of the user. In some embodiments, App 102 is configured to secure, store, and communicate the information in accordance with established data security and privacy standards represented by Electronic Health Record (EHR) guidelines (see https://www.cms.gov/Medicare/E-Health/EHealthRecords/index.html) and Health Insurance Portability and Accountability Act (HIPAA) compliance requirements (see https://www.hhs.gov/hipaa/index.html/; see also https://www.hhs.gov/hipaa/for-professionals/special-topics/cloud-computing/index.html).

Information captured during use of the present invention as combination therapy serves multiple purposes. The combined effects of the present invention and of the pharmacologic agent can be compared to the effects of the respective monotherapies. These comparisons can be made by varying the method of use of the present invention, for example the duration of using a Halo™ system and the time of day of use, the type of relaxation methods used, whether more than one method is used, in the course of managing the muscle tension or headache condition, including whether the system is used as a preventive measure or as an acute therapy at or after the onset of the muscle tension or headache condition. Additional comparisons can be made by recording the combined effect of using a Halo™ system with varying regimens of the pharmacologic agent (including type, dosage, and frequency of administration). Since the present invention and a given pharmacologic agent are individually efficacious, their additive benefits are anticipated. It is also anticipated that any undesirable side effects associated with the pharmacologic agent might be reduced without compromising the therapeutic benefit by reducing the dosage and/or frequency of administration in combination with using the Halo™ system. The extent of those benefits can be characterized and quantified by controlled studies of a given combination therapy, for example in clinical trials. The present invention is uniquely equipped to facilitate such studies and clinical trials.

In one embodiment, components of the wearable device are packaged in the form of a headband. The headband or its functional equivalent is secured to the head of the user by means of adjustable closures or by means of flexible, elastic materials. The sensor array is positioned to contact the skin of the subject at the frontal muscle group (consisting of, but not limited to, the frontalis muscles), typically with a centrally located ground electrode and two working electrodes, one on each side of the ground electrode and located above each eye. The electrodes function without the need for an electrically conductive gel between the electrode surface and where it contacts the surface of the skin. The headband is designed to fit comfortably on the head of the user while exerting uniform pressure on all electrodes to ensure good skin contact with minimal motion artifacts. Other components of the wearable device are housed on a circuit board subassembly integrated into a headband. In other embodiments, the sensor array is built into the headband with integral conductive elements. In other embodiments, the wearable device is in the form of an enclosed, flexible, unitary construction, and protected against solid and liquid intrusion during use. In other embodiments, components of the wearable device are packaged in the form of an audio headset. The sensor array is built into parts of the headset that contact the skin, for example on the earcups. In other embodiments, components of the wearable device are packaged in the form of an earphone or an in-ear monitor, in which sensor(s) are positioned on the peripheral surface of the device where it contacts the skin. In such configurations, the sensors are designed to collect signals primarily from the frontalis, temporalis, and trapezius muscle groups that are strong indicators of muscle tension.

In other embodiments, components of the wearable device are packaged in the form of a neckband. The sensor array is built into parts of the neckband that contacts various regions of the neck and extend from the back of the neck to or beyond the collar bone. Various embodiments of devices with sEMG sensors situated around the neck capture signals related to muscle tension as well as heart rate, breathing patterns, posture, teeth clenching, etc., thereby enabling algorithms to identify and extract signatures associated with specific medical conditions. In other embodiments, sensors or sensor arrays are embedded in various forms of harnesses or apparel: hats, caps, helmets, hard hats, form-fitting shirts, halters, brassieres, chest straps, arm bands, waist bands, leg bands, socks, underwear, gloves, and the like, to collect muscle tension signals from the corresponding parts of the body. Other types of sensors include, but not limited to, HRV, temperature, oximetry, motion detection, and geolocation. These embodiments significantly broaden the scope and capabilities of designs in previous inventions (see Lillydahl, E. and Kirell, A., U.S. Pat. No. 8,690,800, Systems and Methods for Reducing Subconscious Neuromuscular Tensions Including Bruxism).

In some embodiments, multiple sensors are deployed in conjunction with sEMG sensors to provide supplemental or complementary information in the form of additional, discrete “modes.” Such information is used by healthcare professionals to monitor and analyze the conditions of the patient and to support intervention decisions. Thus, in some embodiments, multiple sensing units 200, including different sensor types, are located in one or more wearable devices to collect contemporaneous information from one or more locations of the body, all connected to electronics and algorithm whose multi-mode output delivers different types of diagnostic information. For example, an sEMG sensor situated on the frontal muscle group monitors muscle tension while a thermal sensor placed at an extremity of the body monitors temperature variations in response to stress. In this way, the system of the present invention serves both the user and the healthcare professional through a variety of configurations, information content, and user interfaces to assist in delivering optimal outcome.

Another aspect of the present invention involves features in app 102 that promote user adherence to treatment protocols. These include, but are not limited to, tracking functions that monitor and document system usage; performance metrics showing the condition of the user before and after a given treatment and progress over time; reminders to initiate usage; information on the type and dosage of pharmacologic agents the user may be using either by self-administration or under the guidance of a healthcare provider; and provisions to document user experience. Other features of app 102 include on-demand coaching, provider assistance, and telemedicine functions.

Sensors suitable for sEMG applications are available in various materials of construction. In general, wet electrodes (or dry electrodes with a gel-wetted contact surface to the skin) exhibit low impedance that favors reliable signal capture. Dry electrodes are more convenient in use and in storage, but exhibit much higher impedance, and are susceptible to noise and motion artifacts. Silver/silver chloride is a preferred dry electrode surface because of its stable electrode potential. Other noble metals such as gold also perform well. Hybrid electrodes are commercially available that exhibit the electrochemical characteristic of wet Ag/AgCl electrodes, but feature a dry contact surface. Other conductive materials are suitable in certain embodiments of the wearable device. These include, but are not limited to, materials coated or filled with conductive particles such as silver, chloridized silver, or carbon (graphite, graphene, carbon nanotubes, carbon nanowires, etc.). In other embodiments, conductive ink is used to create electrodes of various sizes, shapes, textures, electrical properties, and on substrates that are rigid or pliable, with optional three-dimensional features that enhance contact, user comfort, and other features that optimize manufacturability and cost. Specific implementation of electronics and algorithm in the present invention enables reliable registration of user signals by means of dry electrodes under various use cases. This is a significant improvement over the prior art based on wet, gel-coated, or composite electrodes constructed from wet and dry components where system performance is acceptable only over a limited range of skin conditions.

Signal processing in the present invention uses algorithms designed to maximize signal-to-noise ratio, and provides sufficient sensitivity to capture minor muscular activity that corresponds to minor increases in tension, and sufficient headroom to avoid saturation with intense muscular activity. In one embodiment, signal processing comprises: 1) an impedance-matched differential analog input stage connected to the sensor array; 2) common-mode rejection to reduce stray electromagnetic interference; 3) multistage analog amplifier; 4) analog-to-digital conversion; 5) modified multivariate wavelet de-noising or functionally equivalent algorithm; and 6) wireless transmission to the computing device.

In an alternate embodiment, signal processing comprises: 1) a sensor array; 2) an analog amplification and signal conditioning subsystem that converts input from the sensors to an output voltage that is proportional to the muscle tension of the wearer; and 3) microcontroller and BLE circuitry that transmit the variable voltage signal and related information wirelessly to the computing device.

In an alternate embodiment, signal processing comprises: 1) a sensor array; 2) an analog amplification subsystem that produces a variable-frequency signal wherein the frequency is modulated by the tension level of the wearer; 3) a frequency-to-voltage converter that generates an output voltage that is proportional to the input frequency; and 4) microcontroller and BLE circuitry that transmit the variable voltage signal and related information wirelessly to the computing device. The variable frequency signal, scaled to span a convenient range in the audible spectrum, provides a means of communicating the intensity of muscle tension to the wearer in addition to, or in the absence of, visual feedback from a computing device.

In either embodiment, signals collected by the sensor array are processed to accurately reflect the intensity of the muscle tension, track changes of the tension level, and communicated to the computing device continuously or at specific intervals. Optionally, when a pre-set threshold of tension level is reached, on-board haptic, audio, and/or visual components are triggered to provide alert feedback to the user directly from the wearable device.

In certain embodiments, the wearable device can be used independent of the computing device to offer a simplified feature set. For example, the device can be set to respond to fixed or pre-set muscle tension thresholds. When this threshold is reached, the on-board user-interface is activated to alert the user to initiate relaxation regimens.

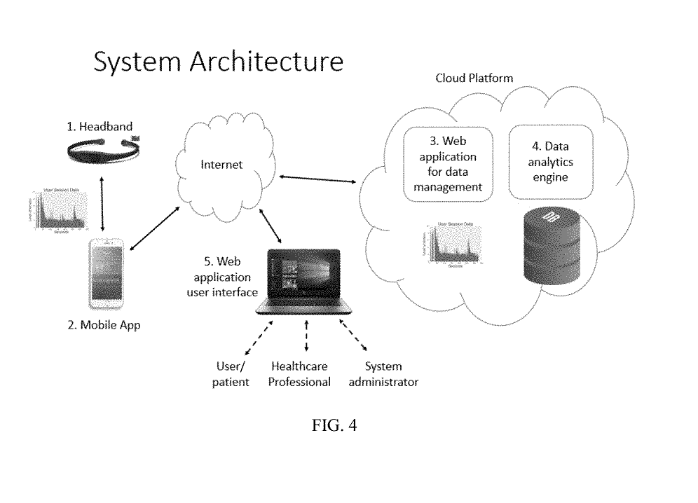

In some embodiments, the present invention can be operated on a stand-alone basis (i.e., sensor output is not transmitted beyond the user's computing device), while in other embodiments, sensor output (whether before or after processing/normalization) can be transmitted to other computing devices (e. g., a server) where it can be analyzed in conjunction with sensor output pertaining to other users. For example, sensor output from many wearable devices can be anonymized, aggregated, and analyzed to reveal trends and to develop treatments/techniques that may be applicable to all or many users. Accordingly, three levels of implementation of the present invention are contemplated: 1) use by individual users (i.e. consumer use); 2) group use (e.g., use by a member of an institutional healthcare system); 3) use by prescription user (e. g., independent patient under the supervision of a healthcare professional such as physician, therapist, counselor, etc.); 4) telemedicine in which the patient communicates with, and receives counsel or treatment from, his/her healthcare provider remotely in accordance with well-established methods and protocols, preferably in real time; and 5) data analytics functions that include, but are not limited to, machine learning capabilities built into processes 251, or linkage with providers such as IBM Watson™, Google Assistant™, and other emerging artificial intelligence data analytics platforms. A general embodiment of the present system architecture thus comprises: 1) a headband to monitor tension; 2) a mobile application to guide the user through a session and gather and upload data; 3) a web data management application to gather and store information upload by each user; 4) an analytics platform to report and improve effectiveness in the application; and 5) a web interface application for users, healthcare professionals, and system administrators to manage information. This network architecture is illustrated in FIG. 4.

As indicated above, the present invention can comprise a device that is designed to be worn on virtually any area of the body to detect excess or desirable muscle tension in that area. In particular, the wearable device can be configured such that, when worn, the array of sensors will be positioned in contact with or in close proximity to the skin overtop the muscles to be monitored. In this way, the present invention can be employed to monitor and treat many chronic pain conditions including, but not limited to: tension headache; migraine; TMJ/MPD (Temporomandibular Joint/Myofascial pain dysfunction); head/neck/shoulder/trapezius pain; chronic pelvic pain (lower abdominal pain); non-disc low back pain; hypercholesterolemia; PTSD; sleep disorders such as apnea or insomnia; bruxism; hypertension; ADD or ADHD; urinary incontinence; alcoholism or substance abuse; arthritis; general chronic pain; fecal elimination disorders; traumatic brain injury; vulvar vestibulitis; headache; cancer pain; back pain, neck pain, pain associated with post-concussion syndrome, etc. In addition, the present invention can be configured in such a way to monitor rehabilitation of damaged muscles and muscles that have experienced nerve damage, for example in patients recovering from physical trauma and multiple sclerosis.

Embodiments of the present invention may comprise or utilize computing devices such as special purpose or general-purpose computers including computer hardware, such as, for example, one or more processors and system memory. Embodiments within the scope of the present invention also include physical and other computer-readable media for carrying or storing computer-executable instructions and/or data structures. Such computer-readable media can be any available media that can be accessed by a general purpose or special purpose computer system.

Computer-readable media is categorized into two disjoint categories: computer storage media and transmission media. Computer storage media (devices) include random access memory (RAM), read-only memory (ROM), electrically-erasable programmable read-only memory (EEPROM), compact disk read-only memory (CD-ROM), solid state drives (SSDs), flash memory, phase-change memory (PCM), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Computer storage also includes network-attached storage (NAS) and cloud-based storage that are accessible locally or remotely (for example over the Internet) relative to the computing device of the present invention. The result of this connectivity is virtually unlimited capacity for the system of the present invention to store and manage information even with relatively simple computing devices. Compared with legacy treatment paradigms, mobile systems of the present invention can reach a much broader population including those with limited means or access to professional care facilities.

Computer-executable instructions comprise, for example, instructions and data which, when executed by a processor, cause a general-purpose computer, special purpose computer, or special purpose processing device to perform a certain function or group of functions. The computer executable instructions may be in the form of programs written in high-level programming languages, or low-level programming code such as, for example, binaries, intermediate format instructions such as assembly language or P-Code, or source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, smartphones, other mobile telephones, personal digital assistants (PDAs), tablets, pagers, routers, switches, and the like.

The invention may also be practiced in distributed system environments where local and remote computer systems, which are linked (either by hardwired data links, wireless data links, or by a combination of hardwired and wireless data links) through a network, including local networks and the Internet, both perform tasks. In a distributed system environment, program modules may be located in both local and remote memory storage devices. An example of a distributed system environment is a cloud of networked servers or server resources. Accordingly, the present invention can be hosted in a cloud environment as shown in FIG. 4. In some embodiments, computing environments and services supporting the present invention are offered and accessed on-demand under the model of “Software as a Service (SaaS),” for example using a thin client via a web interface.

### Methods of Treating Muscle Tension-Related Medical Conditions

A wealth of research has demonstrated that biofeedback therapies are effective for a variety of conditions, most notably behavioral and psychophysiological disorders (see Yucha and Gilbert, 2004. Evidence-Based Practice in Biofeedback and Neurofeedback, https://www.aapb.org/files/public/Yucha-Gilbert_EvidenceBased 2004.pdf). The present invention pertains to a platform technology targeting reduction of stress and muscle tension that underlie a wide variety of medical conditions, and the prevention, mitigation, or resolution of those conditions.

Various embodiments of the present invention include one or more methods of treating a patient having an indication, wherein biofeedback and biofeedback-assisted relaxation therapy are beneficial to treating the indication, and/or a symptom associated with the indication. Various embodiments of the present invention further include one or more methods of treating a patient having an indication, wherein biofeedback and biofeedback-assisted relation therapy alone (monotherapy) or in combination with one or more therapeutic agents (combination therapy).

The potential benefits of combination therapies can be appreciated in the context of current therapies based on pharmacologic agents. Drug development is a constant quest to balance efficacy and side effects, the extent of which depends on patient condition, comorbidities, and other factors. Even the latest formulations are still accompanied by significant adverse reaction profiles (see Press Release, May 12, 2017. Eli Lilly. https://investor.lilly.com/releasedetail.cfm?ReleaseID=1026201; see also MedPage Today, Apr. 26, 2017. https://www.medpagetoday.com/meetingcoverage/aan/64819). To the extent that a combination therapy with the present invention is more efficacious than drug monotherapy, opportunities exist to reduce the dosage of the pharmacologic agent while achieving an equivalent or superior therapeutic outcome depending on the synergy between tension reduction and pharmacologic effects. This is the concept of dose sparing. It is anticipated that a large fraction of pharmaceuticals used in pain management that also carry undesirable side effects are candidates for combination therapy with the present invention. Furthermore, the present invention provides a universal platform to collect, aggregate, and analyze very large amounts of information from mono- or combination therapy, and patient response to assist in dose optimization and dose sparing. With advances in data analytics and artificial intelligence, the present invention could provide fresh insight into drug development and therapeutic approaches beyond the current paradigm.

In one embodiment, the present invention includes a method of treating a patient having an indication by a user wearing the wearable device. A method of treatment further comprises prescribing use of the wearable device. In one embodiment, the present invention includes a method of treating a patient having an indication by the user wearing the device and administering one or more pharmacologic agents intended to treat or prevent the indication. In one embodiment, the present invention includes a method of treating a patient comprising prescribing use of the wearable device, and further prescribing one or more therapeutic agents intended to treat the indication. In one embodiment, a method of treatment of the present invention further comprises administration of a therapeutic agent intended to treat the indication to gain an improved therapeutic outcome. In one embodiment, the patient is instructed to administer the therapeutic agents in response to a user alert from the feedback unit, which occurs in response to sensor data indicating the early onset or presence of an indication. In one embodiment, a method of treatment of the present invention further comprises one or more techniques intended to treat the indication to gain an improved therapeutic outcome. In one embodiment, the patient performs the technique in response to a user alert from the feedback unit. In one embodiment, a method of treatment of the present invention further comprises use or prescription of one or more therapeutic agents and one or more techniques intended to treat the indication to gain an improved therapeutic outcome. In one embodiment, the user is instructed to administer the therapeutic agent and perform the technique in response to a user alert from the feedback unit. In one embodiment, a method of treatment of the present invention further includes prescribing and/or using one or more therapeutic agents at a reduced dosage and/or frequency of administration and/or route of administration and/or duration of administration to reduce the frequency of undesirable side effects associated with one or more of the therapeutic agents.

In some embodiments, the present invention may diagnostically identify whether the user is a candidate for one or more relaxation training techniques. In further detail, in some embodiments, the present invention may detect excessive muscle tension and/or may recommend one or more relaxation training techniques intended to treat the excessive muscle tension. For example, when the output of the sensor unit indicates that the user is experiencing excess tension in one or more muscles or muscle groups, the app 102 may be configured to select or determine the relaxation training techniques intended to treat the excessive muscle tension. In some embodiments, the app 102 may be configured to select or determine the relaxation training techniques based on a characteristic of the excessive muscle tension, such as, for example, one or more of the following: a particular area of the body in which the excessive muscle tension is located, a tension pattern, a tension intensity, and a duration of tension. Additionally, or alternatively, in some embodiments, the app 102 may be configured to select or determine the relaxation training techniques based on sensor data from one or more non-sEMG sensors, such as a time of day associated with tension, an activity or motion associated with tension, or a level of illuminance and/or sound associated with tension.

In some embodiments, the feedback unit of the device of the present invention is configured to create a user alert in response to sensor data. In some instances, a user alert is created prior to the user detecting a physical symptom of an indication (e.g., pain), wherein the indication or a physical symptom indicative of or associated with the indication (e.g., muscle tension) is detectable by one or more sensors of the device. In one embodiment, a user alert informs the user of the presence of a physical symptom that may be indicative of one or more indications. In one embodiment, a user alert informs the user of the onset of one or more indications. In one embodiment, a user alert informs the user of a status of one or more indications or physical symptoms associated with an indication. For example, in one embodiment a user alert indicates that a physical symptom indicative of an indication is steady, increasing, decreasing, or absent. In one embodiment, the wearable device and app of the present invention are configured to detect and alert the user of early onset of an indication and instruct the user to administer one or more agents or therapies prophylactically, thereby preventing complete onset of an indication. In one embodiment, the wearable device and app are configured to monitor diminution of the indication and instructs the user to modify or discontinue further administration of one or more agents to minimize side effects associated with continued administration after the required therapeutic effect has been reached. The ability to dynamically regulate dosage in response to real-time monitoring of an indication offers a level of therapeutic control uniquely enabled with the present invention.

In one embodiment, a user alert instructs the user to administer one or more therapeutic agents intended to reduce, eliminate, or provide relief of a physical symptom of the indication. For example, a user alert may prompt or remind a user to administer an over-the-counter or prescribed medication to reduce muscle tension and/or pain associated with muscle tension. In one instance, a user alert may prompt or remind a user to administer a nutraceutical agent. In one embodiment, a device of the present invention detects a physical symptom, identifies an indication from the physical symptom, and creates a user alert instructing the user to administer a specific therapeutic agent known to treat the indication.

In one embodiment, a user alert instructs the user to initiate one or more techniques intended to reduce, eliminate, or provide relief of a physical symptom of the indication. For example, a user alert may prompt or remind a user to complete a physical therapy exercise or relaxation activity, such as stretching, deep or controlled breathing, progressive muscle relaxation, focused muscle contractions, walking, meditation, eliminating light, changing position, or assuming a body position, such as lying down. In one embodiment, a user alert instructs the user to initiate one or more techniques and one or more therapeutic agents to reduce, eliminate, or provide relief of a physical symptom of an indication.

In some embodiments, when the output of the sensor unit indicates that the user is experiencing excess tension in one or more muscles or muscle groups, the app 102 may be configured to prompt the user to provide additional data via the app 102. The app 102 may use the additional data to determine the relaxation training techniques intended to treat the excessive muscle tension. In some embodiments, the additional data may include, for example, a location of the user, a schedule of the user, health-related information of the user, and/or data regarding the excessive muscle tension, such as a location of the excessive muscle tension, a strength of the excessive muscle tension, etc. In some embodiments, the app 102 can also be configured to process the historical sensor output, the historical samples, and/or the additional data to detect tension patterns or to detect a potential indication.

The following exemplary applications illustrate general and specific methods of use of the present invention.

### Migraine

Migraine is a neurological disease characterized by recurrent, severe headaches and other symptoms that are often debilitating. Only a fraction of patients receive therapy; and most of these receive pharmacologic therapy only. Pharmacologic side effects are common and often cited as the cause for discontinuing prevention therapy. The inventive technology can be used in the treatment of acute headaches (migraine and non-migraine) in combination with one or more of the following medications and for the intended therapeutic benefit of using less of the medication, or using it more safely, or using it more efficaciously, or any combination of the foregoing.

Treatment for acute headaches (migraine and non-migraine) often involves administration of drugs such as onabotulinumtoxinA, topiramate, amitriptyline, propranolol, triptans (such as sumatriptan, zolmitriptan, and rizatriptan), nonsteroidal anti-inflammatory drugs (NSAIDs) (such as aspirin, ibuprofen, naproxen, indomethacin, diclofenac, and ketorolac), acetaminophen, barbiturates (such as butalbital), antidopaminergic drugs (such as metoclopramide), muscle relaxants (such as cyclobenzaprine, methocarbamol, tizanidine, metaxalone, diazepam, and alprazolam), and vasoconstrictors (such as isometheptene). Other pharmacologic agents used for preventive treatment of migraine include anticonvulsants (such as valproate/valproic acid, gabapentin, topiramate, and carbamazine), beta blockers (such as propranolol, atenolol, and metoprolol), serotonin antagonists (such as methysergide), antidepressants (such as amitriptyline, nortriptyline, buspirone, pregabalin), antihistamines (such as diphenhydramine and cyproheptadine). These and other analgesics are used in label, off-label, and over-the-counter (OTC) indications (see http://www.webmd.com/drugs/condition-1116-migraine.aspx?names-dropdown=). This broad selection reflects a diversity of patient conditions and side-effect profiles. Very recent announcements of experimental drugs such as fremanezumab, eptinezumab, galcanezumab and erenumab demonstrate that drugs reduce migraine days effectively, but also exhibit significant side effects. A monotherapy embodiment of the present invention is expected to provide efficacy comparable to current and experimental medications but with no side effects. A combination therapy embodiment of the present invention with, for example, topiramate, amitriptyline, fremanezumab, eptinezumab, galcanezumab, or erenumab, is expected to return greater preventive or therapeutic effects at dosages recommended by the drug innovator companies, or comparable preventative or therapeutic effects at reduced dosages and with fewer side effects. These advantages are especially meaningful for pediatric migraine, where side effects are less well tolerated, and a behavioral therapy, such as biofeedback-assisted, non-invasive approach, is a welcome alternative (see Powers, et al., Cognitive Behavioral Therapy Plus Amitriptyline for Chronic Migraine in Children and Adolescents: A Randomized Clinical Trial, JAMA. 2013 Dec. 25; 310(24): 2622-2630; and Kroner, et al., Cognitive Behavioral Therapy plus Amitriptyline for Children and Adolescents with Chronic Migraine Reduces Headache Days to <4 Per Month, Headache 2016:56:711-716).

Opioids (such as codeine, oxycodone, hydrocodone, morphine, meperidine, tramadol, and hydromorphone) are powerful analgesics that are used to treat migraine when other medications fail or are ineffective. However, these chemicals carry serious side effects, with risks of developing tolerance, dependence, medication overuse headache, and narcotic-induced hypersensitivity. (see https://migraine.com/blog/risks-of-long-term-opioid-treatment/; and https://migraine.com/blog/recommended-guidelines-for-opioid-treatment/). Over the course of a decade, opioid abuse and opioid use disorder (OUD) have become a national crisis in the United States (see https://wayback.archive-it.org/8315/2017 0119081343/https://www.hhs.gov/blog/2015/12/10/rates-of-drug-overdose-deaths-continue-to-rise.html). These dynamics highlight the urgent need for non-narcotic, and indeed non-pharmacological, interventions exemplified by the present invention.

### Epilepsy, Dementia, Alzheimer's Disease

Epilepsy and migraine are highly comorbid chronic neurologic disorders. Their clinical presentation, risk factors, mechanisms, and treatments overlap (see Epilepsy Foundation web site: http://www.epilepsy.com/information/professionals/co-existing-disorders/migraine-epilepsy; see also Silberstein, S. D. and Lipton, R. B., Headache and epilepsy. In: Ettinger A B and Devinsky O, eds. Managing epilepsy and co-existing disorders. Boston: Butterworth-Heinemann; 2002; 239-254). To the extent that the present invention has demonstrated efficacy in resolving migraine and other headaches, the same system and its underlying technology are expected to show efficacy toward epileptic conditions. Accordingly, the inventive technology can be used in the treatment of one or more neurologic disorders in combination with one or more of the following medications and for the intended therapeutic benefit of using less of the medication, or using it more safely, or using it more efficaciously, or any combination of the foregoing.

Considerable research has been focused on illnesses of the brain. There are studies suggesting association between migraine and dementia (see http://migraine.newlifeoutlook.com/migraine-and-dementia/; see also http://www.health.harvard.edu/mind-and-mood/migraines-can-dementia-stroke-or-heart-attack-be-next) and Alzheimer's disease (see http://ispub.com/IJH/8/2/11263) based on certain similarities in cerebral alterations. With further investigation, the present invention is poised to help relieve the symptoms or slow progression of these neurological impairments.

In one embodiment, the present invention comprises a therapy for treatment of seizure indications, comprising use of the wearable device. The present invention further comprises a therapy for treatment of seizure indications comprising prescribing to a patient use of the wearable device as a monotherapy. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for seizure indications further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of seizure indications that are treatable by the methods of the present invention include epilepsy, generalized seizures, absence seizures, focal seizures, simple focal seizures, complex focal seizures, and secondary generalized seizures. Non-limiting examples of therapeutic agents for treatment of seizure indications that are compatible with the methods of the present invention include brivaracetam, carbamazepine, diazepam, lorazepam, clonazepam, eslicarbazepine, ethosuximide, felbamate, lacosamide, lamotrigine, levetiracetam, oxcarbazepine, perampanel, phenobarbitol, phenytoin, pregabalin, tiagabine, topiramate, valproate, and zonisamide.

### Orofacial Pain (Including Temporomandibular Joint Disease (DV))

The inventive technology may further be used in the treatment of orofacial pain in combination with one or more of the following medications and for the intended therapeutic benefit of using less of the medication, or using it more safely, or using it more efficaciously, or any combination of the foregoing.

In one embodiment, the present invention comprises a therapy for treatment of pain and/or inflammatory indications, comprising use of the wearable device. The present invention further comprises a therapy for treatment of pain and/or inflammatory indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for pain and/or inflammatory indications further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of pain and/or inflammatory indications that are treatable by the methods of the present invention include back pain, headache, toothache, muscular aches, pelvic pain, menstrual cramps, arthritis, common cold, flu, sinus pain, jaw pain, neck pain, shoulder pain, bursitis, sprains, inflammatory disease, osteoarthritis, rheumatoid arthritis, gout, tendonitis, fibromyalgia, and primary dysmenorrhea. Non-limiting examples of therapeutic agents for treatment of pain and/or inflammatory indications that are compatible with the methods of the present invention include paraaminophenols, salicylates, propionic acid derivatives, indoleacetic acids, benzothiazine derivatives, pyrroleacetic acid derivatives, triptans, tricyclics antidepressants, and COX-2 inhibitors.

### Insomnia

An expert review conducted by the American Sleep Disorders Association in 2006 of nonpharmacological treatments of insomnia concluded that psychological and behavioral interventions were effective in the treatment of chronic insomnia (see Morgenthaler, T., Kramer, M., Alessi, C., Friedman, L., Boehlecke, B., Brown, T., et al. (2006). Practice parameters for the psychological and behavioral treatment of insomnia: An update. An American Academy of Sleep Medicine Report. Sleep, 29(11), 1415-1419). A subsequent review of nonpharmacologic options that included relaxation therapy, biofeedback, and cognitive behavioral therapy (i.e., various types of psychotherapy in which negative patterns of thought about the self and the world are challenged in order to alter unwanted behavior patterns or treat mood disorders) further supported these approaches, particularly when medications are not indicated, as an augmentation to medication, or as individual therapy in short-term mild insomnia (see Morin, A. K., Jarvis, C. I., & Lynch, A. M. (2007). Therapeutic options for sleep-maintenance and sleep onset insomnia. Pharmacotherapy, 27(1), 89-110). These findings and recommendations are consistent with the present invention as monotherapy and combination therapy, with the added benefit of eliminating side effects in the case of combination therapy.

The inventive technology may further be used in the treatment of insomnia with one or more of the following medications and for the intended therapeutic benefit of using less of the medication, or using it more safely, or using it more efficaciously, or any combination of the foregoing. Non-limiting examples of therapeutic agents for treatment of insomnia (i.e., sleep onset and/or sleep maintenance) include: suvorexant; eszopiclone; zaleplon; zolpidem; triazolam; temazepam; ramelteon; doxepin; trazodone; tiagabine; diphenhydramine; melatonin; tryptophan; and valerian (see Sateia M J, Buysse D J, Krystal A D, Neubauer D N, Heald J L. Clinical practice guideline for the pharmacologic treatment of chronic insomnia in adults: an American Academy of Sleep Medicine clinical practice guideline. J Clin Sleep Med. 2017; 13(2):307-349). Each drug is a candidate for combination therapy to enhance an already significant improvement in sleep quality observed in the course of developing the present invention.

In one embodiment, the present invention comprises a therapy for treatment of sleep-related indications, comprising use of the wearable device. The present invention further comprises a therapy for treatment of sleep-related indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for sleep-related indications further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of sleep-related indications that are treatable by the methods of the present invention include sleep apnea, insomnia, circadian rhythm disorders, restless leg syndrome, and narcolepsy. Non-limiting examples of therapeutic agents for treatment of sleep-related indications that are compatible with the methods of the present invention include dopamine agonists, benzodiazepines, non-benzodiazepine hypnotics, melatonin receptor simulators, opiates, anticonvulsants, anti-narcoleptics, and orexin receptor antagonists.

### Chronic Pain (Including Chronic Pelvic Pain and Pelvic Floor Pain (Dyspareunia))

In one embodiment, the present invention comprises a therapy for treatment of pelvic floor pain, comprising use of the wearable device. The present invention further comprises a therapy for treatment of pelvic floor pain comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for pelvic floor pain further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of chronic pain in combination with one or more of the following medications and for the intended therapeutic benefit of using less of the medication, or using it more safely, or using it more efficaciously, or any combination of the foregoing. Non-limiting examples of therapeutic agents for treatment of pelvic floor pain that are compatible with the methods of the present invention include birth control pills, progestin, gonadotrophin-releasing hormone agonists, NSAIDs, tricyclic antidepressants, laxatives, and anticonvulsants.

In one embodiment, a combination therapy for treatment of pelvic floor pain further comprises prescribing to a patient a therapeutic device or procedure intended to treat pelvic floor pain. Non-limiting examples of therapeutic devices or procedures for treatment of pelvic floor pain that are compatible with the methods of the present invention include physical therapy, physical activity, and dietary restrictions.

### Multiple Sclerosis

Multiple sclerosis (MS) is an unpredictable, often disabling disease of the central nervous system. Symptoms can be relieved, and disease progression delayed, but no cure exists at this time. Immune modulators can be administered to reduce the frequency and severity of attacks. Common medications include interferon beta 1-b, and a range of biopharmaceuticals (see http://www.webmd.com/drugs/condition-1078-Multiple+Sclerosis). A recent review pointed to the risks of MS-related stress leading to stress-related disorders such as anxiety and depression, and pointed to the limitations of symptomatic drug-based therapies, and the practice of mind-body medicine as especially helpful when psychosocial stress is a factor or non-pharmacological options are desired (e.g. during pregnancy) (see Senders, A., Wahbeh, H., Spain, R., and Shinto, L., Mind-Body Medicine for Multiple Sclerosis: A systematic Review, Autoimmune Diseases, 2012, Article ID 567324). Significantly, muscle relaxants were reported to be helpful for reducing stress-triggered new MS lesions, anxiety and depression, and to help spasticity, an issue in many cases of MS (see Mohr D C, Hart S L, Julian L, Cox D, Pelletier D. Association between stressful life events and exacerbation in multiple sclerosis: a meta-analysis. BMJ 2004; 328: 731). This strongly suggests that relaxation through biofeedback by means of the present invention would provide similar outcomes, and to do so without side effects associated with drugs.

The inventive technology may further be used in the treatment of multiple sclerosis with one or more of the following medications and for the intended therapeutic benefit of using less of the medication, or using it more safely, or using it more efficaciously, or any combination of the foregoing. Specifically, the present invention offers three areas of applicability: 1) controlling stress to reduce incidence of new MS lesions; 2) relieving anxiety and depression associated with MS; and 3) reducing spasticity. Combination therapy embodiments of the present invention include, but are not limited to, Avonex (interferon beta-1a), Betaseron (interferon beta-1b), Copaxone (glatiramer acetate), Extavia (interferon beta-1b), Glatopa (glatiramer acetate), Plegridy (peginterferon beta-1a), Rebif (interferon beta-1a), Zinbryta (daclizumab), Aubagio (teriflunomide), Gilenya (fingolimod), Tecfidera (dimethyl fumarate), Lemtrada (alemtuzumab), Novantrone (mitoxantrone), Ocrevus (ocrelizumab), Tysabri (natalizumab). Each combination therapy is expected to return greater therapeutic effects at the same dosages of the drug used alone, or to deliver comparable therapeutic effects at reduced dosages and with fewer side effects.

### Tinnitus

In one embodiment, the present invention comprises a therapy for treatment of tinnitus, comprising use of the wearable device. The present invention further comprises a therapy for treatment of tinnitus comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for tinnitus further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of tinnitus with one or more of the following medications and for the intended therapeutic benefit of using less of the medication, or using it more safely, or using it more efficaciously, or any combination of the foregoing. Non-limiting examples of therapeutic agents for treatment of tinnitus that are compatible with the methods of the present invention include anti-anxiety drugs, antidepressants, steroids, misoprostol, lidocaine, and various herbal preparations.

In one embodiment, a combination therapy for treatment of tinnitus further comprises prescribing to a patient a therapeutic device or procedure intended to treat tinnitus. Non-limiting examples of therapeutic devices or procedures for treatment of tinnitus that are compatible with the methods of the present invention include tinnitus retraining therapy, a sound masking device, cognitive therapy, dental treatment, cochlear implants, and acupuncture.

### Gastrointestinal Diseases

The inventive technology may further be used in the treatment of various gastrointestinal diseases with one or more of the following medications and for the intended therapeutic benefit of using less of the medication, or using it more safely, or using it more efficaciously, or any combination of the foregoing.

### Crohn's Disease

Stress adversely affects a person's normal digestive process. Crohn's disease results from inflammation of the bowel. Flare-ups are triggered or symptoms worsen as stress levels increase. Conventional medical treatment are aimed at reducing inflammation that triggers symptoms by means of anti-inflammatory drugs such as oral 5-aminosalicylates (sulfasalazine [Azulfidine] and mesalamine [Asacol, Delzicol, Pentasa, Lialda, Apriso]), which are no longer in widespread use because numerous side effects including nausea, diarrhea, vomiting, heartburn, and headache; and corticosteroids (e.g. prednisone, budesonide), which also have serious side effects especially with extended use or only effective in specific bowel locations. Immunosuppressants such as azathioprine (Imuran) and mercaptopurine (Purinethol) are more widely used but require close monitoring by physicians. Other immune system suppressors include TNF inhibitors such as infliximab (Remicade), adalimumab (Humira), and certolizumab pegol (Cimzia). These drugs cannot be used for people with certain conditions, with the risk of serious complications. Other potent medications are sometimes used where the patient does not respond to other treatments. These include methotrexate (Rheumatrex), cyclosporine (Gengraf, Neoral, Sandimmune), tacrolimus (Astagraf XL, Hecoria), natalizumab (Tysabri), Vedolizumab (Entyvio), and ustekinumab (Stelara). Antibiotics such as metronidazole (Flagyl) or ciprofloxacin (Cipro) are also administered in cases where bacterial infection is a concern.

Studies at the Mayo Clinic have identified biofeedback, relaxation and breathing exercises to be effective stress-reduction techniques to control symptoms and lengthen the time between flare-ups (see Mayo Clinic website: http://www.mayoclinic.org/diseases-conditions/crohns-disease/basics/lifestyle-home-remedies/con-20032061). Accordingly, the present invention in the form of monotherapy can be deployed as a first-line treatment free from side effects, or in conjunction with diet and exercise as a safer form of combination therapy than medications, or used as combination therapy with medications cited above at reduced dosages to mitigate adverse reactions.

Inflammatory Bowel Disease and/or Irritable Bowel Syndrome

In one embodiment, the present invention comprises a therapy for treatment of inflammatory bowel disease (“IBD”) and/or irritable bowel disease (“MS”), comprising use of the wearable device. The present invention further comprises a therapy for treatment of IBD and/or IBS comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for IBD and/or IBS further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of IBD- and/or IBS-related indications that are treatable by the methods of the present invention include ulcerative colitis, Crohn's disease, collagenous colitis, diverticulitis, and lymphocytic colitis. Non-limiting examples of therapeutic agents for treatment of IBD and/or IBS that are compatible with the methods of the present invention include immunosuppressive drugs, steroids, NSAIDs, antibiotics, probiotics, and various herbal therapies.

In one embodiment, a combination therapy for treatment of IBD and/or IBS further comprises prescribing to a patient a therapeutic device or procedure intended to treat these indications. Non-limiting examples of therapeutic devices or procedures for treatment of IBD and/or IBS that are compatible with the methods of the present invention include proctocolectomy, physical activity, counseling, acupuncture, and dietary restrictions.

### Constipation

In one embodiment, the present invention comprises a therapy for treatment of constipation, comprising use of the wearable device. The present invention further comprises a therapy for treatment of constipation comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for constipation further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of therapeutic agents for treatment of constipation that are compatible with the methods of the present invention include laxatives, increased or decreased fiber intake, stimulants, osmotics, lubricants, stool softeners, lubiprostone, linaclotide, lactulose, and polyethylene glycol.

In one embodiment, a combination therapy for treatment of constipation further comprises prescribing to a patient a therapeutic device or procedure intended to treat constipation. Non-limiting examples of therapeutic devices or procedures for treatment of constipation that are compatible with the methods of the present invention include physical therapy, pelvic muscle training, physical activity, and dietary restrictions.

### Anxiety

Many studies support the efficacy of behavioral modification therapy including biofeedback in treating anxiety. Biofeedback is generally regarded as being safer than drugs. Monotherapy based on the present invention addresses the same symptoms of anxiety and should thus be equally effective as other biofeedback methods. Interestingly, a controlled study in 1977 first reported that combining EMG biofeedback with the drug diazepam (Valium) effectively reduced muscular tension. Results also indicated that EMG feedback treatment without diazepam had a more prolonged therapeutic effect for chronic anxious patients (see Yvon Jacques Lavallée, Yves Lamontagne, Gilbert Pinard, Lawrence Annable, Léon Tétreault, Effects on EMG feedback, diazepam and their combination on chronic anxiety Journal of Psychosomatic Research, 1977 Volume 21, Issue 1, Pages 65-71).

The inventive technology may further be used in the treatment of anxiety with one or more of the following medications and for the intended therapeutic benefit of using less of the medication, or using it more safely, or using it more efficaciously, or any combination of the foregoing. The present invention used as monotherapy or combination therapy with diazepam will not only realize the promise of that early work for many, but also significantly reduce the risks, including well-documented abuse, of the popular drug. The selection of drugs to treat anxiety conditions have since expanded significantly. A first group of drugs are commonly used to treat general anxiety disorders, including but limited to Lexapro; Cymbalta; Effexor XR; citalopram; Paxil CR; escitalopram; quetiapine; sertraline; Paxil; venlafaxine; paroxetine; pregabalin; duloxetine; Pexeva; and Irenka (see https://www.drugs.com/condition/generalized-anxiety-disorder.html). A second group of drugs are used to treat anxiety and stress, including but not limited to Celexa; Prozac; sertraline; citalopram; fluoxetine; Paxil; amitriptyline; venlafaxine; paroxetine; Prozac Weekly; Luvox CR; Luvox; prazosin; and fluvoxamine (see https://www.drugs.com/condition/anxiety-stress.html). Each drug from these lists is a candidate for use as combination therapy with the present invention.

In one embodiment, the present invention comprises a therapy for treatment of anxiety indications, comprising use of the wearable device. The present invention further comprises a therapy for treatment of anxiety indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for anxiety indications further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of anxiety indications that are treatable by the methods of the present invention include panic disorders, social anxiety disorders, and generalized anxiety disorders. Non-limiting examples of therapeutic agents for treatment of anxiety indications that are compatible with the methods of the present invention include antidepressants, selective serotonin reuptake inhibitors (SSRIs), antihistamines, and beta-blockers.

### Depression

The inventive technology may further be used in the treatment of depression with one or more of the following medications and for the intended therapeutic benefit of using less of the medication, or using it more safely, or using it more efficaciously, or any combination of the foregoing.

Many people take antidepressants such as Paxil, Zoloft, and Prozac to treat depression. These are drugs known as selective serotonin reuptake inhibitors (SSRIs).

In one embodiment, the present invention comprises a therapy for treatment of depression indications, comprising use of the wearable device. The present invention further comprises a therapy for treatment of depression indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for depression indications further includes prescribing and/or using one or more therapeutic agents at a reduced dosage and/or frequency of administration and/or route of administration and/or duration of administration to reduce the frequency of undesirable side effects associated with one or more of the therapeutic agents.

Non-limiting examples of depression indications that are treatable by the methods of the present invention include major depression, persistent depressive disorder, bipolar disorder, seasonal affective disorder (SAD), psychotic depression, postpartum depression, premenstrual dysphoric disorder (PMDD), situational depression, and atypical depression. Non-limiting examples of therapeutic agents for treatment of depression indications that are compatible with the methods of the present invention include selective serotonin reuptake inhibitors (SSRIs), serotonin and norepinephrine reuptake inhibitors (SNRIs), norepinephrine and dopamine reuptake inhibitors (NDRIs), atypical antidepressants, tricyclic antidepressants, and monoamine oxidase inhibitors (MAOIs).

### Attention Deficit Hyperactivity Disorder (ADHD)

Certain types of SSRIs are also prescribed to treat attention deficit hyperactivity disorder (ADHD). Although these drugs are effective and generally safe when used as prescribed, patients should be aware of potential side effects. For example, administering an SSRI for orofacial pain may lead to worsening of the pain. In some cases the patient experienced bruxism, broken teeth, and headaches after taking an SSRI (see Ferguson, J M, SSRI Antidepressant Medications: Adverse Effects and Tolerability, Primary Care Companion J Clinical Psychiatry 3:1, February 2001, 22-27). Given the effectiveness of biofeedback for these conditions, any drug regimen for anxiety, depression, and ADHD can be used with the present invention as combination therapy.

In one embodiment, the present invention comprises a therapy for treatment of attention deficit hyperactivity disorder (“ADHD”) and/or attention deficit disorder (“ADD”), comprising use of the wearable device. The present invention further comprises a therapy for treatment of ADHD/ADD comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for ADHD/ADD further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of ADD and/or ADHD with one or more of the following medications and for the intended therapeutic benefit of using less of the medication, or using it more safely, or using it more efficaciously, or any combination of the foregoing. Non-limiting examples of therapeutic agents for treatment of ADD and/or ADHD indications that are compatible with the methods of the present invention include stimulants, amphetamine, dextroamphetamine, lisdexamfetamine, methylphenidate, atomoxetine, clonidine, guanfacine, amitriptyline, desipramine, imipramine, nortiptyline, tricyclic antidepressants, bupropion, escitalopram, sertraline, and venlafaxine.

### Cervical Vertigo, Disequilibrium and Dizziness, Globus (Functional Dysphagia)

The inventive technology may further be used in the treatment of Cervical vertigo with one or more of the following medications and for the intended therapeutic benefit of using less of the medication, or using it more safely, or using it more efficaciously, or any combination of the foregoing.

Cervical vertigo is a common condition among head and neck muscle tension syndromes, often resulting in dizziness and disequilibrium. The usual treatment involves administrating cyclobenzaprine (Flexeril) or amitriptyline. These are antidepressants often rejected by patients due to their side effects. The present invention is a safe alternative to treat cervical vertigo-related conditions. Another condition that affects 40% of the population is globus, a persistent or intermittent feeling of a lump in the throat when the person is under stress. Anti-depressants and cognitive behavioral therapy are effective treatments (see Bong Eun Lee and Gwang Ha Kim, Globus pharyngeus: A review of its etiology, diagnosis and treatment, World J Gastroenterol. 2012 May 28; 18(20): 2462-2471). The present invention offers a modern, accessible tool for patients to manage these conditions without drugs.

### Hypertension

The relationship between stress and hypertension is well recognized. Several studies including controlled clinical trials and meta-analyses confirm that biofeedback via various means (HRV, biofeedback-based training with active interventions such as relaxation and meditation) resulted in reductions in blood pressure comparable to active treatment with pharmaceuticals (see Nolan, R. P., et al, Hypertension 2010; 55:1033-1039). A monotherapy embodiment of the present invention is expected to provide blood pressure reductions comparable to but with no side effects. A combination therapy embodiment of the present invention in conjunction with current hypertension medications is expected to return greater preventative or therapeutic effects at dosages used in the trials by the drug innovator companies, or deliver comparable preventative or therapeutic effects at reduced dosages accompanied by reduced side effects.

In one embodiment, the present invention comprises a therapy for treatment of hypertension indications, comprising use of the wearable device. The present invention further comprises a therapy for treatment of hypertension indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for hypertension indications further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of hypertension with one or more of the following medications and for the intended therapeutic benefit of using less of the medication, or using it more safely, or using it more efficaciously, or any combination of the foregoing. Non-limiting examples of hypertension indications that are treatable by the methods of the present invention include malignant hypertension, secondary hypertension, and renal hypertension. Non-limiting examples of therapeutic agents for treatment of hypertension indications that are compatible with the methods of the present invention include thiazide diuretics, calcium channel blockers, ACE inhibitors, angiotensin II receptor antagonists (ARBs), and beta blockers.

### Hyperlipidemia Indications

In one embodiment, the present invention comprises a therapy for treatment of hyperlipidemia indications, comprising use of the wearable device. The present invention further comprises a therapy for treatment of hyperlipidemia indications comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for hyperlipidemia indications further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of hyperlipidemia indications with one or more of the following medications and for the intended therapeutic benefit of using less of the medication, or using it more safely, or using it more efficaciously, or any combination of the foregoing. Non-limiting examples of therapeutic agents for treatment of hyperlipidemia indications that are compatible with the methods of the present invention include statins, fibrates, niacin, bile acid sequestrants, ezetimibe, lomitapide, phytosterols, and orlistat.

### Urinary Incontinence

In one embodiment, the present invention comprises a therapy for treatment of fecal and/or urinary incontinence, comprising use of the wearable device. The present invention further comprises a therapy for treatment of incontinence comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for incontinence further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of incontinence with one or more of the following medications and for the intended therapeutic benefit of using less of the medication, or using it more safely, or using it more efficaciously, or any combination of the foregoing. Non-limiting examples of therapeutic agents for treatment of incontinence that are compatible with the methods of the present invention include anticholinergics, myrbetriq, alpha blockers, topical estrogen, injections of botulinum toxin type A, anti-diarrheals, laxatives, injectable bulking agents, increased fluid intake, and increased intake of high-fiber foods. In one embodiment, a combination therapy for treatment of incontinence further comprises prescribing to a patient a therapeutic device or procedure intended to treat incontinence. Non-limiting examples of therapeutic devices or procedures for treatment of incontinence that are compatible with the methods of the present invention include physical therapy, bowel training, sacral nerve stimulation, posterior tibial nerve stimulation, vaginal balloon, sphincteroplasty, treatment of rectal prolapse, a rectocele or hemorrhoids, sphincter replacement or repair, and colostomy.

### Post-Traumatic Stress Disorder

In one embodiment, the present invention comprises a therapy for treatment of post-traumatic stress disorder (“PTSD”), comprising use of the wearable device. The present invention further comprises a therapy for treatment of PTSD comprising prescribing to a patient use of the wearable device. In one embodiment, the present invention comprises a combination therapy that further comprises prescribing and/or using with the wearable device one or more therapeutic agents intended to treat the indication to gain an improved therapeutic outcome. In one embodiment, the patient is instructed to administer the therapeutic agent in response to a user alert from the wearable device or a feedback unit associated with the wearable device. In one embodiment, a method of treating a patient for PTSD further includes prescribing and/or using one or more therapeutic agents at a reduced dosage and/or frequency of administration and/or route of administration and/or duration of administration to reduce the frequency of undesirable side effects associated with one or more of the therapeutic agents.

The inventive technology may further be used in the treatment of PTSD with one or more of the following medications and for the intended therapeutic benefit of using less of the medication, or using it more safely, or using it more efficaciously, or any combination of the foregoing. Non-limiting examples of therapeutic agents for treatment of PTSD that are compatible with the methods of the present invention include selective serotonin reuptake inhibitors (“SSRIs”), sertraline, paroxetine, paroxetine mesylate, escitalopram, venlafaxine, citalopram, fluoxetine, amitriptyline, mirtazapine, and fluvoxamine. In one embodiment, a combination therapy for treatment of PTSD further comprises prescribing to a patient a therapeutic device or procedure intended to treat PTSD. Non-limiting examples of therapeutic devices or procedures for treatment of PTSD that are compatible with the methods of the present invention include counseling, physical activities, a companion animal, cognitive behavioral therapy, cognitive processing therapy, and prolonged exposure therapy.

### Miscellaneous

Other embodiments of the present invention can be employed to monitor and treat conditions including, but not limited to, interstitial cystitis, dysmenorrhea, fibromyalgia, reflex sympathetic dystrophy (RSD), “urethral syndrome,” and vulvar vestibulitis.

The exemplary applications above and the considerable supporting research illustrate the extraordinary benefits of the present invention based on a versatile, accessible mobile technology platform that can be deployed stand-alone or to enhance the latest advances in pharmaceutical development to improve patient outcome. Being non-invasive, side effect-free and drug-agnostic, the invention has virtually universal applicability for conditions caused or exacerbated by muscle tension syndromes.

### EXAMPLES

### Example 1

This example shows the results of a pilot study completed in April 2017. The objective of the study was to determine the effects of using the present invention on headache frequency and intensity on human subjects.

Six subjects were enrolled in the New York City area with varying headache conditions (such as tension headaches, migraines, and TMJ (temporomandibular joint) pain. The study began with a four-week baseline period during which subjects recorded the frequency and intensity of their headache condition. This was followed by a four-week treatment period, in which subjects were instructed to use Halo™ headbands and app each day for a period of 15 minutes, recording their pain conditions and other use experiences. Headache frequency was reported as episodes per week. Headache intensity was subjectively reported on a standard scale of 0-10 (see Loder et al., Measuring pain intensity in headache trials: which scale to use? Cephalalgia 32(3) 2012, 179-182). Results are shown below in Table 1:

TABLE 1

| | |
|---|---|
| Headache Frequency | Headache Intensity |

| Subject | Baseline | Treated | Baseline | Treated |
|---|---|---|---|---|
| 10001 | 4 | 1 | 4 | 1 |
| 10002 | 3 | 1 | 2.5 | 3 |
| 10004 | 3 | 1 | 6 | 3 |
| 10005 | 8 | 1 | 8 | 1 |
| 10006 | 10 | 2 | 6 | 6 |
| 10011 | 3 | 3 | 4 | 4 |

Overall, five subjects out of six reported a reduction of headache frequency with treatment using Halo™; five subjects out of six reported a reduction of headache intensity; all subjects reported a reduction in muscle tension; four subjects out of six reported an improvement in mood; two out of three subjects who used over-the-counter or prescription pain medication reported a significant decrease in the use of the medication; and all subjects reported improved sleep quality.

### Example 2

This example shows results from evaluation of a prototype mobile system of the present invention conducted on a human subject. The subject was a 28-year old American female in New York City with a history of recurring headaches. Tests were conducted in three stages between December 2016 and March 2017. In the first stage the subject used various over-the-counter analgesics at recommended dosages to manage headache symptoms (Sessions 1 and 2). Conditions were self-assessed prior to drug administration according to a standard headache scale, and then qualitatively reassessed for the extent of relief. In the second stage, the subject took an analgesic at the same time of using a prototype Halo™ system for 5 min (Session 3). In the third stage the subject used the prototype Halo™ system alone for a duration of 10 min on three occasions (Sessions 4-6). Results are shown below in Table 2:

TABLE 2

| | HA Intensity | | | |
|---|---|---|---|---|
| | prior to | | Halo (TM) | |
| Session | treatment* | Drug treatment | treatment | Relief |
| 1 | 9 | ibuprofen + naproxen | | No relief |
| 2 | 6 | aspirin | | Moderate |
| 3 | 6 | naproxen | 5 min | Complete |
| 4 | 2 | — | 10 min | Complete |
| 5 | 3 | — | 10 min | Complete |
| 6 | 1 | — | 10 min | Complete |

These results suggest that analgesics alone was effective for controlling headache to various extents but not reliably for this subject. Combining the use of Halo™ system with the strongest analgesic in the group produced complete relief of moderate headache (Session 3). Using Halo™ alone appeared to relieve mild headaches quite effectively.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed is:

1. A system for detecting muscle tension, comprising:
a wearable device having at least one sensor unit configured to generate at least one output indicative of a level of muscle tension, and circuitry for processing the output.

2. The system of claim 1, wherein processing the output comprises detecting when the output exceeds a threshold thereby indicating that a user of the wearable device is experiencing undesirable muscle tension.

3. The system of claim 2, wherein the undesired muscle tension is excessive muscle tension or inadequate muscle tension.

4. The system of claim 2, wherein processing the output further comprises providing an alert when the output exceeds the threshold.

5. The system of claim 4, wherein the alert is one or more of a haptic alert, a visual alert, or an audible alert.

6. The system of claim 1, wherein processing the output comprises transmitting the output to an app executing on a computing device.

7. The system of claim 6, further comprising:
the app that is configured to receive the output transmitted by the circuitry of the wearable device and process the output to produce a real-time display indicative of the level of muscle tension.

8. The system of claim 7, wherein the mobile or app is further configured to detect when the level of muscle tension exceeds a threshold and in response notify a user of the wearable device.

9. The system of claim 8, wherein notifying the user includes presenting one or more treatment techniques that the user can perform to reduce the muscle tension.

10. The system of claim 9, wherein the one or more treatment techniques include one or more relaxation techniques, one or more cognitive behavioral therapy techniques, and/or one or more pharmacologic agents.

11. The system of claim 9, wherein the app is further configured to monitor the output after presenting the one or more treatment techniques to verify whether the one or more treatment techniques have reduced the muscle tension.

12. The system of claim 7, wherein the mobile or app is configured to store the output over time and to analyze the stored output to detect patterns or trends in muscle tension.

13. The system of claim 7, wherein the mobile or app is configured to transmit the output to one or more remote computing devices, servers, or cloud based storage system.

14. The system of claim 13, wherein the one or more remote computing devices, servers, or cloud based storage system include a computing device, servers, or cloud based storage system of a healthcare provider with which the user has a relationship.

15. The system of claim 14, wherein the mobile or computing device is configured to receive communications from a user of the one or more remote computing devices, servers, or cloud based storage system and to display the communications to the user of the wearable device, the communications including instructions for addressing the muscle tension.

16. The system of claim 13, wherein the one or more remote computing devices, servers, or cloud based storage system include a computing device that receives output generated by a plurality of wearable devices worn by other users.

17. The system of claim 7, wherein the mobile or app is further configured with telecommunication features to connect with a remote clinician.

18. A method for treating an indication, comprising use of the system of any of claims 1 through 17.

19. The method of claim 18, further comprising administration of one or more pharmacologic agents, and/or one or more cognitive behavioral therapies intended to treat the indication.

20. The method of either claim 18 or 19, wherein the indication is selected from the group consisting of pain, inflammation, anxiety, depression, sleep-related disorders, hypertension, seizure, hyperlipidemia, ADHD, ADA, IBD, IBS, constipation, pelvic floor pain, incontinence, PTSD, and tinnitus.

21. The method of any of claims 18 through 20, further comprising administering the one or more pharmacologic agents in response to an alert from the wearable device.

22. The method of claim 21, wherein the step of administering further comprises modifying a regimen of the one or more pharmacologic agents to initiate, reduce, or terminate use of the pharmacologic agents.

23. The method of any of claims 18 through 21, further comprising administering at least one of a physical activity, physical therapy, a relaxation technique, a dietary restriction, counseling, a companion animal, cognitive behavioral therapy, cognitive processing therapy, and prolonged exposure therapy.

24. A system for detecting an indication, comprising:
a wearable device having a sensor unit configured to detect one or more biological signals of the indication, the sensor unit further configured to generate an output indicative of the indication, and circuitry for processing the output.

25. The system of claim 24, wherein processing the output comprises detecting when the output exceeds a threshold thereby indicating that a user of the wearable device is experiencing the one or more biological signals indicating undesirable muscle tension.

26. The system of claim 25, wherein the one or more biological signals is selected from the group consisting of muscle tension, heart rate, temperature, EEG, EKG/ECG, GSR, HRV, and pulse oximetry.

27. The system of claim 24, wherein the indication is selected from the group consisting of tension headache, migraine, TMJ/MPD, muscle pain, chronic pelvic pain, non-disc low back pain, hypercholesterolemia, PTSD, apnea, insomnia, bruxism, hypertension, ADD, ADHD, urinary incontinence, alcoholism, substance abuse, arthritis, chronic pain, fecal elimination disorders, traumatic brain injury, vulvar vestibulitis, epilepsy, dementia, Alzheimer's disease, multiple sclerosis, tinnitus, Crohn's disease, inflammatory bowel disease, constipation, anxiety, depression, vertigo, hyperlipidemia, and pain associated with cancer and post-concussion syndrome.

28. A method of treating an indication of claim 27 in a patient in need thereof that comprises (i) administration of an effective amount of at least one pharmacologic agent, and (ii) prescribing use of the system of any of claims 1 through 27.

29. The method of claim 28, wherein the indication is a migraine.

30. The method of claim 29, wherein the pharmacologic agent is selected from the group consisting of an analgesic, an NSAID, acetaminophen, barbiturates, antidopaminergic drugs, muscle relaxants, vasoconstrictors, anticonvulsants, beta blockers, serotonin antagonists, antidepressants, antihistamines, topiramate, amitriptyline, propranolol, fremanezumab, eptinezumab, galcanezumab, and erenumab, and other monoclonal antibodies peptides, biomolecules, and small molecule drugs for treating migraines.

31. The method of claim 28, wherein the indication is multiple sclerosis.

32. The method of claim 31, wherein the pharmacologic agent is selected from the group consisting of natalizumab, ocrelizumab, alemtuzumab, daclizumab, teriflunomide, fingolimod, mitoxantrone, other biologicals such as interferon beta-1a, interferon beta-1b, peginterferon beta-1a, and other small-molecule drugs, for treating multiple sclerosis.

33. The method of claim 28, wherein the indication is acute and chronic pain conditions.

34. The method of claim 33, wherein the pharmacologic agent is selected from the group consisting of analgesics, NSAIDs, opioids, and other biologicals or small-molecule drugs for pain management and treatment.

35. The method of claim 28, wherein the indication is sleep disorder, including insomnia, sleep apnea, circadian rhythm disorders, restless leg syndrome, and narcolepsy.

36. The method of claim 35, wherein the pharmacologic agent is selected from the group consisting of dopamine agonists, benzodiazepines, non-benzodiazepine hypnotics, melatonin receptor simulators, opiates, anticonvulsants, anti-narcoleptics, orexin receptor antagonists, suvorexant, eszopiclone, zaleplon, zolpidem, triazolam, temazepam, ramelteon, doxepin, trazodone, tiagabine, diphenhydramine, melatonin, tryptophan, valerian, and other drugs for treating sleep disorders.

37. The method of claim 28, wherein the indication is anxiety.

38. The method of claim 37, wherein the pharmacologic agent is selected from the group consisting of anti-anxiety drugs, antidepressants, steroids, misoprostol, lidocaine, Cymbalta, Effexor XR, citalopram, Paxil CR, escitalopram, quetiapine, sertraline, Paxil, venlafaxine, paroxetine, pregabalin, duloxetine, Pexeva, Irenka, Celexa, Prozac, sertraline, citalopram, fluoxetine, amitriptyline, venlafaxine, paroxetine, Prozac Weekly, Luvox CR, Luvox, prazosin, fluvoxamine, and other drugs for treating anxiety.

39. The method of claim 28, wherein the indication is a neurologic disorder, comprising at least one of epilepsy, dementia, and Alzheimer's disease.

40. The method of claim 39, wherein the pharmacologic agent is selected from the group consisting of brivaracetam, carbamazepine, diazepam, lorazepam, clonazepam, eslicarbazepine, ethosuximide, felbamate, lacosamide, lamotrigine, levetiracetam, oxcarbazepine, perampanel, phenobarbitol, phenytoin, pregabalin, tiagabine, topiramate, valproate, zonisamide, and other biologicals and small-molecule drugs for treating neurologic disorders.

41. The method of claim 28, wherein the indication is depression.

40. The method of claim 41, wherein the pharmacologic agent is selected from the group consisting of Avonex (interferon beta-1a), Betaseron (interferon beta-1b), Copaxone (glatiramer acetate), Extavia (interferon beta-1b), Glatopa (glatiramer acetate), Plegridy (peginterferon beta-1a), Rebif (interferon beta-1a), Zinbryta (daclizumab), Aubagio (teriflunomide), Gilenya (fingolimod), Tecfidera (dimethyl fumarate), Lemtrada (alemtuzumab), Novantrone (mitoxantrone), Ocrevus (ocrelizumab), Tysabri (natalizumab), Paxil, Zoloft, Prozac, and other biologicals or small-molecule drugs for treating depression.

41. The method of claim 28, wherein said indication is selected from the group consisting of tension headache, migraine, TMJ/MPD, muscle pain, chronic pelvic pain, non-disc low back pain, hypercholesterolemia, PTSD, apnea, insomnia, bruxism, hypertension, ADD, ADHD, urinary incontinence, alcoholism, substance abuse, arthritis, chronic pain, fecal elimination disorders, traumatic brain injury, vulvar vestibulitis, epilepsy, dementia, Alzheimer's disease, multiple sclerosis, tinnitus, Crohn's disease, IBD, constipation, anxiety, depression, vertigo, hyperlipidemia, and pain associated with cancer and post-concussion syndrome.

42. The method of claim 28, wherein the indication manifests at least one biological signal detectable by the system.

43. The method of claim 42, wherein the at least one biological signal is selected from the group consisting of muscle tension, heart rate, temperature, EEG, EKG/ECG, GSR, HRV, and pulse oximetry.

44. The method of claim 28, further comprising alerting the patient via an output of the system.

45. The method of claim 44, wherein the output comprises at least one of an audible signal, a visual signal, and a haptic signal.

46. The method of claim 45, further comprising alerting the patient via an output of the system of at least one of (i) the presence of the at least one biological signal, (ii) the presence of the indication, (iii) a reminder, (iv) coaching, and (v) a remedial action for the indication.

47. The method of claim 46, wherein at least one of the reminder, the coaching, and the remedial action for the indication alerts the patient to complete at least one of (i) administer the pharmacologic agent, (ii) complete a physical therapy exercise, (iii) complete a relaxation activity, and (iv) other forms of cognitive behavioral therapy.

48. The method of claim 47, wherein the physical therapy exercise or relaxation exercise comprises at least one of stretching, deep breathing, controlled breathing, progressive muscle relaxation, focused muscle contractions, walking, meditation, eliminating light, changing position, and assuming a body position.

49. The method of any of claims 28 through 48, further comprising administration of at least one of a physical activity, physical therapy, a relaxation technique, a dietary restriction, counseling, a companion animal, cognitive behavioral therapy, cognitive processing therapy, and prolonged exposure therapy.
